(19) United States
(12) Patent Application Publication
(10) Pub. No.: US 2004/0202726 A1
(43) Pub. Date: Oct. 14, 2004

(54) Topical blood pressure composition

(76) Inventor: Samuel L. DeShay, Columbia, MD (US)

Correspondence Address: SHELDON & MAK, INC, 225 SOUTH LAKE AVENUE, 9TH FLOOR, PASADENA, CA, 91101, US

(21) Appl. No.: 10/412,080

(22) Filed: Apr. 10, 2003

(51) Int. Cl. A61K 33/14

(52) U.S. Cl. 424/679

(57) ABSTRACT

A topical composition useful for reducing blood pressure having a liquid carrier containing water, potassium chloride, and a penetrant enhancer for enhancing the penetration of the potassium chloride into the skin.

### BACKGROUND

The present invention relates to a topical composition, and more particularly to a topical composition for treating and preventing high blood pressure.

Hypertension is a disease affecting many people. Hypertension is generally defined as an abnormally high blood pressure. It is clinically recognized as an elevation of systolic arterial blood pressure of 140 mm Hg or higher and/or an elevation of diastolic arterial blood pressure of 90 mm Hg or higher. High blood pressure is thought to lead to a variety of ailments and to an increased likelihood of death from heart attack, stroke, and heart failure.

It is common to treat hypertension using drugs. However, drug therapy should be reserved for individuals whose blood pressure cannot be maintained in an acceptable range by non-pharmacological means. It is also known that weight reduction, limiting alcohol, and the restriction of dietary sodium may in some instances reduce hypertension. Some individuals find weight loss and the restriction of dietary sodium to be very difficult.

Long term studies suggest that the amount of potassium in the diet may also be an important determinant of blood pressure in patients with hypertension. It has been observed that increasing dietary potassium intake tends to lower blood pressure. However, the demand of increasing dietary potassium intake conflicts with the goals of weight loss and the restriction of dietary sodium. Some have proposed dietary supplements containing potassium. See for example, U.S. Pat. Nos. 5,498,428 and 5,804,204 to Morris, Jr. et al. However, some individuals are adverse to taking dietary supplements in the form of a pill.

Therefore, there is a need for a way of supplementing an individual's potassium intake without pharmaceuticals or dietary supplements. The present invention fulfills this and other related needs.

### SUMMARY OF THE INVENTION

The present invention is directed to a topical composition useful for reducing blood pressure having a water and oil mixture, potassium chloride, and a penetrant enhancer for increasing the penetration of the potassium chloride into the skin. Optionally, the penetrant enhancer can be at least one of glyceryl stearate and PEG 100 stearate. The topical composition can contain an emollient such as one or more of isopropyl myristate, lanolin alcohol and cetyl alcohol.

The topical composition may contain: a lubricant such as one or more of dimethicone, mineral oil, and stearyl alcohol; a hydrating agent such as aloe vera and propylene glycol; an antioxidant such as vitamin E acetate; a preservative such as one or more of methyl paraben and propyl paraben; a fragrance such as lavender; and/or at least one of olive oil, Hawthorne berry extract and garlic extract.

The present invention is also directed to a method for treating high blood pressure where the composition is applied to the skin.

### DESCRIPTION

In accordance with the present invention, a novel topical composition is provided for preventing or treating hypertension. The composition contains potassium and a penetrant to increase absorption of the potassium through the skin. The following descriptions represent various embodiments of the present compositions. These embodiments are not intended to limit the scope of the invention.

The amount of each ingredient in the composition is given in weight percent based on the total weight of the composition. Such ingredients can be synthetically prepared and/or derived from natural sources. Further, the compositions of the present invention may comprise, consist of or consist essentially of the ingredient's identified herein. Embodiments disclosed herein can be formulated by combining, adding, mixing and/or blending the herein described ingredients (e.g., in commercial grade form) using standard techniques known in the art.

A topical composition according to a preferred embodiment of the present invention contains potassium chloride. The potassium chloride is the active ingredient designed to be absorbed through the skin into the bloodstream to lower blood pressure. Additionally, the potassium chloride functions to increase the viscosity of the composition.

In addition to the potassium chloride, the composition contains a penetrant enhancer. In one version of the present invention, the composition contains a mixture of glyceryl stearate and PEG 100 stearate known as Lexemul 561 as a penetrant enhancer. However, either glyceryl stearate or PEG 100 stearate could be used individually as a penetrant enhancer. In addition to functioning as penetrant enhancers, both glyceryl stearate and PEG 100 stearate are emulsifiers. Glyceryl stearate also acts as a skin lubricant and imparts a pleasant skin feel. Glyceryl stearate is also a solvent, humectant, and consistency regulator in water-in-oil and oil-in-water formulations. PEG 100 stearate also acts as a cleansing agent and surfactant.

Additional emulsifiers and emollients may be added to the composition. One additional emollient that may be added is isopropyl myristate which also acts as a moisturizer and skin softener that also assists in product penetration. Another emulsifier and emollient that may be added is lanolin alcohol which absorbs a considerable amount of water that is then slowly released for moisturization purposes. An additional emulsifier and emollient that may be added is cetyl alcohol which also acts as a thickener.

Additional ingredients may be added as lubricants to impart lubricity, slip, and good feel. An additional ingredient to impart lubricity is dimethicone, which can also serve as a formulation defoamer. Dimethicone also improves product flow and spreadability. Another ingredient that may be added to impart lubricity is mineral oil which also acts as a protective agent and binder. Another ingredient that may be added to impart lubricity is olive oil, which has also been reported lower blood pressure. Yet another ingredient that may be added to impart lubricity is stearyl alcohol which can also serve as a formulation defoamer and for increasing the viscosity of the composition.

Optionally, a hydrating and softening, agent may be added. An exemplary hydrating and softening additive is aloe vera, which supplies moisture directly to the skin. An additional hydrating agent that may be added is propylene glycol. Optionally, an antioxidant may be added. An exemplary antioxidant is vitamin E acetate which also acts as a skin moisturizer. A preservative such as methyl paraben and/or propyl paraben may also be added.

Optionally, fragrance may be added to the composition to impart a pleasant odor to a user's skin. An exemplary fragrance is lavender, which is soluble in most fixed oils.

Optionally, Hawthorne berry extract (crataegius laevigata) may be added to provide additional therapeutic properties to the composition. Hawthorne berry extract is believed to act as an antispasmodic, as a vasodilator, and as a sedative. Hawthorne berry extract useful in the present invention is sold by Nature's Answer, Inc., Hauppauge, N.Y. 11788-3943. Additionally, liquid aged garlic extract (allium sativum) may be added as a therapeutic agent in small amounts. Aged garlic extract that may be used in the present invention is sold by Wakunaga of America Co., Ltd. Mission Viejo, Calif. 92691. It is believed that the addition of the Hawthorne berry extract and the liquid aged garlic extract may function to enhance the effectiveness of the potassium chloride to reduce blood pressure.

The chart below lists the ingredients of one version of the present invention, and the range of amounts of each ingredient. The amounts are given in weight percent based on the total weight of the composition.

| | Approximate | Approximate |
|---|---|---|
| | Minimum Amount | Maximum Amount |
| Ingredient | (Weight %) | (Weight %) |
| Potassium Chloride | 10 | 25 |
| Glyceryl stearate and PEG | 8 | 10 |
| 100 stearate | | |
| Stearyl Alcohol | 3 | 4 |
| Cetyl Alcohol | 2 | 3 |
| Isopropyl Myristate | 2 | 3 |
| Olive Oil | 12 | 14 |
| Dimethicone | <1 | <1 |
| Vitamin E Acetate | <1 | <1 |
| Deionized Water | 54 | 60 |
| Propylene Glycol | 3 | 6 |
| Hawthorne Berry Extract | <1 | 1 |
| (crataegius laevigata) | | |
| Liquid Aged Garlic | <1 | 1 |
| extract (allium sativum) | | |
| Aloe Vera | 3 | 5 |
| Fragrance | <1 | <1 |
| Preservative | 1 | <2 |

The present composition is preferably in the form of a cream formed by a water and oil emulsion. However, the composition but may be formed into a liquid solution, lotion, ointment, salve, spray or gel using conventional methods known in the art. For example, gel based vehicles are well known in the art and commercially available for formulation with active ingredients to form a suitable topical application. Also, when used in gel form, a gelling agent may be present in an amount sufficient to provide the appropriate viscosity.

The present compositions may also be formulated into lotions or salves using methods known in the art. For example, such lotions or salves may be formulated with an aqueous or oily base and will include also, in general, one or more of the following: stabilizing agents, emulsifying agents, dispersing agents, suspending agents, thickening agents, coloring agents, perfumes, and the like.

The present composition may also be in the form of an aerosol using methods known in the art. For example, aerosol formulations for use with the present composition would typically include propellants, surfactants and co-solvents and may be filled into conventional aerosol containers that are closed by a suitable metering valve.

The present invention is also directed to a method for reducing high blood pressure through the application of the composition detailed above. In a preferred embodiment, the method comprises the step of applying a composition having the ingredients discussed above on the skin. The composition may be applied to any portion of the body, but the legs, abdomen and chest are preferred. The composition is applied at least once per day, depending on the potassium chloride in the composition, to give a maximum dosage of 1000 mg of potassium chloride per day. Application of the composition is repeated until blood pressure is reduced to a desired level. In a preferred embodiment, the composition comprises ???? and is applied once per day for fourteen days.

### EXAMPLE 1

### Effect of the Composition of the Present Invention in Patients not on Blood Pressure Medication Prior to the Study

A study was performed on nine people with elevated blood pressure levels. None of the participants were on blood pressure medication prior to commencement of the study. The ages of the participants ranged from 43 to 72, with an average age of 57. The participants were all African American. Three participants were female and six were male.

The participants were given a skin cream. The cream contained the following ingredients as a percentage of the cream by weight: deionized water (65.10%), potassium chloride (10.00%), Lexemul 561 (5.00%), mineral oil (8.00%), propylene glycol (3.00%), stearyl alcohol (3.00%), isopropyl myristate (3.00%), cetyl alcohol (1.00%), dimethicone(0.50%), lanolin alcohol (0.50%), fragrance (0.50%), methyl paraben (0.20%), propyl paraben (0.10%), vitamin E acetate (0.10%), aged garlic extract (0.01%), and Hawthorne berry extract (0.001%).

The participants were instructed to apply 15 grams (about one level tablespoon) of the cream on their abdomen or chest once per day for 15 consecutive days. Blood pressure was measured at the conclusion of the 15 day period and compared to the initial blood pressure measurements. Blood pressure was measured with a Tycos Sphygmomanometer. The average decrease in systolic pressure, was 22.7 mm Hg. The average decrease in diastolic pressure was 8.6 mm Hg.

### EXAMPLE 2

### Effect of the Composition of the Present Invention in Patients not on Blood Pressure Medication Prior to the Study

A study was performed on 11 people with elevated blood pressure levels. All of the participants were on some blood pressure medication, but not controlled, at the time the study was initiated. The ages of the participants ranged from 43 to 66, with an average age of 57. The participants were all African American. Eight participants were female and three were male. Blood pressure measurements were taken with a Tycos Sphygmomanometer.

The participants were given a skin cream. The cream contained the following ingredients as a percentage of the cream by weight: deionized water (65.10%), potassium chloride (10.00%), Lexemul 561 (5.00%), mineral oil (8.00%), propylene glycol (3.00%), stearyl alcohol (3.00%), isopropyl myristate (3.00%), cetyl alcohol. (1.00%), dimethicone(0.50%), lanolin alcohol (0.50%), fragrance (0.50%), methyl paraben (0.20%), propyl paraben (0.10%), vitamin E acetate (0.10%), aged garlic extract (0.01%), and Hawthorne berry extract (0.001%).

The participants were instructed apply 15 grams (about one level tablespoon) of the cream on their abdomen or chest once per day for 15 consecutive days. Blood pressure was measured at the conclusion of the 15 day period and compared to the initial blood pressure measurements. Blood pressure was measured with a Tycos Sphygmomanometer.

Eight participants showed a decrease in systolic pressure, while two participants showed a constant systolic pressure and one participant showed an unexplained increase in systolic pressure. The average systolic pressure decrease in the eight participants showing a decrease was 15 mm Hg. Six participants showed a decrease in diastolic pressure, while one participant showed a constant diastolic pressure and four participants had a rise in Diastolic pressure. The average diastolic pressure decrease in the six participants showing a decrease was 11.2 mm Hg.

Although the present invention has been described in considerable detail with reference to certain preferred versions thereof, other versions are possible. Therefore, the spirit and scope of the appended claims should not be limited to the description of the preferred versions described herein.

Any element in a claim that does not explicitly state “means” for performing a specified function or “step” for performing a specified function, should not be interpreted as a “means” or “step” clause as specified in 35 U.S.C. § 112.

What is claimed is:

1. A topical composition useful for reducing blood pressure comprising:
a liquid carrier comprising:
water;
potassium chloride; and
a penetrant enhancer for enhancing the penetration of the potassium chloride into the skin.

2. The topical composition of claim 1 wherein the penetrant enhancer is glyceryl stearate.

3. The topical composition of claim 1 wherein the penetrant enhancer is PEG 100 stearate.

4. The topical composition of claim 1 wherein the penetrant is a mixture of glyceryl stearate and PEG 100 stearate.

5. The topical composition of claim 1 further comprising an emollient.

6. The topical composition of claim 5 wherein the emollient is at least one of isopropyl myristate, lanolin alcohol and cetyl alcohol.

7. The topical composition of claim 1 further comprising a lubricant.

8. The topical composition of claim 7 wherein the lubricant is at least one of dimethicone, mineral oil, olive oil, and stearyl alcohol.

9. The topical composition of claim 1 further comprising a hydrating agent.

10. The topical composition of claim 9 wherein the hydrating agent is at least one of aloe vera and propylene glycol.

11. The topical composition of claim 1 further comprising an antioxidant.

12. The topical composition of claim 11 wherein the antioxidant is vitamin E acetate.

13. The topical composition of claim 1 further comprising a preservative.

14. The topical composition of claim 13 wherein the preservative is at least one of methyl paraben and propyl paraben.

15. The topical composition of claim 1 further comprising fragrance.

16. The topical composition of claim 15 wherein the fragrance is lavender.

17. The topical composition of claim 1 further comprising at least one of Hawthorne berry extract and garlic extract.

18. A topical composition to reduce blood pressure comprising:
a water and oil emulsion; potassium chloride, isopropyl myristate, glyceryl stearate, PEG 100 stearate, olive oil, lanolin alcohol, dimethicone, vitamin E acetate, methyl paraben, propyl paraben, garlic extract, Hawthorne berry extract and fragrance.

19. A topical composition for treating high blood pressure comprising by weight percent of the total weight of the composition:
a) more than about 10% and less than about 25% potassium chloride;
b) more than about 8% and less than about 10% glyceryl stearate and PEG 100 stearate for enhancing the penetration of the potassium chloride into the skin;
c) more than about 3% and less than about 4% stearyl alcohol;
d) more than about 2% and less than about 3% cetyl alcohol;
e) more than about 2% and less than about 3% isopropyl myristate;
f) more than about 12% and less than about 14% olive oil;
g) less than about 1% dimethicone;
h) less than about 1% vitamin E acetate;
i) more than about 54% and less than about 60% deionized water;
j) more than about 3% and less than about 6% propylene glycol;
k) less than about 1% Hawthorne berry extract;
l) less than about 1% garlic extract;
m) more than about 3% and less than about 5% aloe vera;
n) less than about 1% fragrance; and
o) more than about 1% and less than about 2% preservative.

20. A method for treating high blood pressure comprising the step of applying a composition comprising a liquid carrier comprising water, potassium chloride and a penetrant enhancer to the skin.

21. The method of claim 20 wherein the method further comprises applying the composition to at least one of a leg, chest and abdomen.

22. The method of claim 20 wherein an amount of the composition containing less than about 1000 mg of potassium chloride is applied per day.

23. The method of claim 22 further comprising the step of measuring blood pressure; and wherein application of the composition and measuring of blood pressure are repeated until the measured blood pressure is below a predetermined pressure.
